(19) United States
(12) Patent Application Publication
(10) Pub. No.: US 2025/0241627 A1
(43) Pub. Date: Jul. 31, 2025

(54) OCULAR SUCTION DEVICE

(71) Applicant: HOPITAL FONDATION ADOLPHE DE ROTHSCHILD, PARIS (FR)

(72) Inventor: Eric GABISON, CHARENTON LE PONT (FR)

(21) Appl. No.: 18/702,261

(22) PCT Filed: Oct. 26, 2022
(86) PCT No.: PCT/FR2022/052036

§ 371 Date: Apr. 17, 2024

(30) Foreign Application Priority Data
Oct. 26, 2021 (FR) 2111359

(51) Int. Cl. A61B 10/00

(52) U.S. Cl. CPC A61B 10/0045; A61B 2010/008

(57) ABSTRACT

The invention relates to a method and a device for collecting a sample of interstitial fluid from the eye of an animal, for analysis. The device includes an airtight enclosure whose proximal surface in direct contact with the mucosa has an opening defining a collection area, and whose distal surface comprises at least one port connected to a first suction system, and a collection system consisting of a tube joined to the top of the distal surface of the sealed enclosure, inside which a reservoir slides that is passed through by a perforation system oriented toward the mucosa, opposite the opening. The walls of the sealed enclosure are hollow, and optionally connected to a second suction system.

### TECHNICAL FIELD

The present invention relates to a device for collecting a sample of interstitial fluid from a mucosa, for example the oral or ocular mucosa, of an animal (e.g. a human) for analysis, and a method for using this device.

The present invention is for example applicable in the field of ophthalmology.

### STATE OF THE ART

In dermatology, suction tools are used to create a suction bubble on the skin from which interstitial fluid can be collected. One example is a steel/plexiglass capsule with a hole of defined diameter (4, 8, 11 or 14 mm) at the base, connected to a vacuum pump which applies a slight vacuum of 200 to 300 mmHg to the skin surface. After around 2 hours, interstitial cutaneous fluid accumulates between the dermis and the epidermis, gradually detaching and forming a bubble known as a suction bubble. Another example is the Cutomètre®, which is designed to measure the biomechanical properties of the upper layers of the epidermis by applying a negative pressure (suction) that deforms the skin whereupon it is applied, so as to draw it into a measuring cup of the probe before releasing it inside the probe. The penetration depth is determined by a contactless optical measuring system composed of a light source and a light receiver.

There are also suction tools used in eye surgery, such as Lasik. For example, a suction ring is placed on the surface of the eye and, by means of suction along its periphery (vacuum), secures itself to the eye to enable precise cutting of a “cap” of the cornea by another device (microkeratome) sliding on a rail forming the upper surface of the suction ring. Due to its morphology, the suction ring can only access the contour of the eye, but not the surface of the eye, which is covered from the outside vs. the central area (window) accessible from the outside.

In addition, various types of ocular samples can be collected from the surface of the eye (conjunctival imprint), from inside the eye (aqueous humor) and from tears. Conjunctival imprinting involves applying a “blotter” to the surface of the eye to collect conjunctival epithelial cells in a noninvasive or minimally-invasive, rapid and virtually painless way, for biological analysis of ocular surface diseases. This apposition can be carried out via an instrument, notably Eyeprim™ (single-use), operating on the principle of a syringe: a thumb-operated plunger pushes a membrane over the eye, and when the plunger is released, this membrane returns to a closed container and can then be collected at the end of analysis using forceps.

Patent application US 2016/0235392 discloses a device comprising a system for liquefying tissue associated with a rotating system for abrading said tissue, to cause one or more components of said tissue to be dissolved and collected.

### DESCRIPTION OF THE INVENTION

The inventors have developed a new device for collecting an interstitial fluid sample from a mucosa (that is, a membrane lining the body's cavities (digestive tract, mouth, nasal cavities, bronchi, anus etc.) which connects with the skin at the natural orifices, and which is lubricated by mucus secretion; e.g. oral or conjunctival mucosa), in particular the ocular or conjunctival mucosa, enabling, by creating a suction/aspiration area on the surface of an animal's mucosa, e.g. the conjunctiva (that is, the transparent mucous membrane that lines the inside of the eyelids and the white of the eye in the bulbar part thereof), collection of a sample of interstitial fluid (that is, the fluid between the blood and the cells, whose presence promotes interaction between the two) for analysis thereof.

The device of the invention comprises a hollow volume, hereinafter referred to as the enclosure, which is relatively airtight and inside which a first vacuum can be created. Preferably, the enclosure has transparent parts, notably the top face (distal surface furthest from the mucosa, e.g. from the conjunctiva) and the bottom face (proximal surface in contact with the mucosa, e.g. the conjunctiva) so that the operator can see their operating field. The enclosure may optionally have magnification means on the apex of its distal surface to enable more precise positioning of the enclosure on the surface of the mucosa, e.g. the conjunctiva. The surface of the enclosure in contact with the mucosa, e.g. the conjunctiva (proximal surface), is partially recessed to have an opening (“window”) defining a collection area. Preferably, this surface is substantially curved and bordered by a material, preferentially flexible, serving as a seal between the enclosure and the anterior surface of the mucosa, e.g. of the conjunctiva, in order to adapt in particular to the average curvature of the eye. Different models may exist to suit different eye sizes (in particular child/adult). The window surface, preferentially circular, can have different sizes depending on the device, generally between 3 and 350 mm². Preferably, the window is built on an iris structure enabling the operator, for example, to adjust the diameter of the window by turning the base of the enclosure. The seal bordering the partially recessed surface is in contact with the mucosa, e.g. the conjunctiva. To help with sealing, a cream or other suitable material can be applied to the mucosa, e.g. the conjunctiva, along the seal. This seal can extend substantially in the plane of the partially recessed surface outside the enclosure, as well as inside the enclosure, where it can help define the solid area of the partially recessed surface. The enclosure is connected via a tubing to a system for adjusting the air pressure in the enclosure. This system for example consists of a vacuum pump and a means of evaluating the pressure inside the enclosure, such as a pressure gauge (see FIG. 1). Other known systems—manual, mechanical, etc.—can also be used to create an initial vacuum in the enclosure. For example, a syringe-type, manually-operated plunger-based system can be used to create this vacuum, such that the syringe body can be graduated to indicate the vacuum levels generated in the enclosure. Precise, reactive control and adjustment of the vacuum gradually created in the enclosure is important, particularly given the sensitivity and fragility of the eye. Alternatively, the system creating the vacuum in the enclosure can be a Vacutainer®-type device. The advantage of such a system is that it requires only a relatively simple set of components, notably without a vacuum pump, which can then be manufactured as a single-use pack. In this mode, vacuum control can be achieved by means of a system of valves connecting the Vacutainer® to the device and/or a pressure level in the Vacutainer® which, in relation to the volume of the enclosure, presents no risk to the subject while creating the desired vacuum. Settings, standard stages, successive adjustments and progressive modes of vacuum build-up can be defined/automated to facilitate/standardize the operator's work and take into account the subject's comfort. The walls of the enclosure are hollow and define a space, in contact with the mucosa, e.g. the conjunctiva, around the periphery of the window, within which a second vacuum can be created using the first vacuum system or an additional system (e.g. vacuum pump, Vacutainer® or the like). The walls of the enclosure may or may not communicate with the interior of the enclosure. In the first case, the vacuum inside the enclosure walls and inside the enclosure are identical. The second vacuum created inside the walls of the enclosure enables the mucosa, e.g. the conjunctiva, to be secured along the periphery of the window, thus defining an area whose central part, inside the window, is subjected to the first vacuum created inside the enclosure. This prevents the first vacuum inside the window from leading, due to the relatively high elasticity of mucosa (e.g. conjunctival tissue), to the displacement of too much mucosa, e.g. conjunctiva, inside the enclosure, which would risk potentially lining the inner walls of said enclosure, that is, an excessive distribution of the overpressure so that the first vacuum can cause swelling of part of the mucosa, e.g. the conjunctiva, that is, a suction “bubble” of interstitial fluid to form inside the enclosure.

Thus, once the device according to the invention is applied to the surface of the mucosa, e.g. of the conjunctiva: (i) the first vacuum created inside the enclosure made possible by the action of the vacuum system connected to the enclosure and the sealing of the enclosure due to the seal (not mandatory) ensures solidarity of the enclosure with the mucosa, e.g. the conjunctiva, as well as a relative overpressure of the part of the mucosa, e.g. the conjunctiva, in contact with the device, in particular at the window, for the formation of an interstitial fluid “bubble,” and (ii) the second vacuum created inside the walls of the enclosure ensures that it is completely sealed and secures the mucosa, e.g. the conjunctiva, so as to urge it onto a surface lower than the surface of the periphery of the device, resulting in a localized pressure increase which, without the risk of overpressure (intraocular in the case of the conjunctiva), significantly reduces the time needed for the “bubble” to form. This relative/local overpressure created by the device of the invention therefore gradually creates a “bubble” at the part of the mucosa, e.g. the conjunctiva, inside the enclosure, the outer surface of which bubble is the mucosa, e.g. the conjunctiva, and the interior of which is the interstitial fluid which can then be collected. To this end, the device has a means of collecting a sample at the part of the mucosa, e.g. the conjunctiva, that is accessible via the window, and in particular of taking advantage of the accessibility of the interstitial fluid in the “bubble” due to the relative/local overpressure. Preferably, the interstitial fluid is collected from the “bubble” by means of additional suction at the window, using a system based on the Vacutainer® principle, for example. Under this principle, the device comprises a hollow needle capable of passing through the top of the enclosure, pointing toward the window and such that inside said needle is a one-way valve/gate so that a gas/liquid can only pass through the needle in the direction from inside the enclosure to the outside of the enclosure. Such a valve also prevents outside air from entering the container when generating the vacuum in the enclosure, and reciprocally allows the passage of the element collected by the needle to the outside of the enclosure. This needle has two sharpened ends and can be moved along its axis without compromising the sealing of its connection to the enclosure. The needle can thus be brought closer to the part of the mucosa, e.g. the conjunctiva (suction bubble), passing through the window, or even penetrate this part to the depth desired by the operator. The other end of the needle can then be used, in the manner of a Vacutainer® system, to pierce a reservoir placed in a vacuum such that the internal pressure of said reservoir is lower than the pressure inside the enclosure, creating a suction phenomenon of the collection toward the reservoir. To achieve this, the enclosure has an area at its top (distal surface), made for example made of foam/elastomer, that can be easily pierced by a needle similar to the one shown above, so that it closes around the needle without allowing air through at the working pressures of the device of the invention. A collection means may consist of a tube joined to the top of the enclosure, inside which a reservoir with a needle passing through it can slide. This tube is hermetically sealed and is such that, at rest (off), the needle does not pass through the top of the enclosure. The pressure of the reservoir and the tube in the resting state is such that, when the reservoir is slid toward the enclosure (on), and the needle thus passes through the top of the enclosure and subsequently through the mucosa, e.g. the conjunctiva, forming the outer surface of the suction “bubble,” then the reservoir pressure is equal to Pr, lower than the pressure Pe inside the enclosure, which creates a movement of the interstitial fluid in the suction “bubble” toward the inside of the reservoir. This assumes that when the suction “bubble” is created, the top of the latter is very close to, or even in contact with, the top of the enclosure, in order to limit the vacuum generated by the needle when the latter is inside the enclosure but not yet inside the suction “bubble” (see FIG. 2A). The entire device, with the exception of the vacuum-generating system(s), is for single use only. The operator can have as many needles as reservoirs, and proceed as described above (pierce the top of the enclosure, bring the needle tip toward the part of the mucosa, e.g. the conjunctiva, pass through the window, pierce the reservoir to collect interstitial fluid). The advantage of this collection method is that there is no contamination from one reservoir to another due to needle sharing, should the operator wish to collect several samples (e.g. at different depths). Other known “perforation/suction” systems can also be used for collection (e.g. syringe, etc.). For such a collection device, the path length of the perforation/suction system (needle) is preferentially such that the tip cannot go beyond the lower part of the device and therefore beyond the protuberance formed by the suction bubble. For example, in the case of a needle passing through a sliding reservoir, for safety reasons the length of the collection needle as it exits the reservoir is preferentially less than the height of the walls of the enclosure blocking the path of the reservoir.

Interstitial fluid collection using the device of the invention enables novel analyses to be carried out: biochemical examinations (e.g. pH, osmolarity and ionograms); inflammatory cell research; pharmacokinetic/dynamic studies, for example on eye drops or molecules administered systemically (both surface and depth); chemotherapy side effects (previously studied in tears). It also enables immediate “Lab on a Chip” analyses to study the concentration of various proteins in the interstitial fluid. The device of the invention can also take advantage of the binding force created by suction to collect a full-thickness conjunctival or epithelial (e.g. oral) impression tissue sample of the mucosa from the area in contact with the mucosal surface, e.g. the conjunctiva. In addition, the space created by the device of the invention, particularly under the conjunctiva, can enable substances to be injected into a more limited space than is currently possible with subconjunctival injections.

The present invention therefore relates to a device for collecting a sample of interstitial fluid from the mucosa of an animal for analysis, said device comprising:
(i) an airtight enclosure whose proximal surface in direct contact with the mucosa has an opening defining a collection area, and whose distal surface comprises at least one port connected to a first suction system (to create a vacuum inside the airtight enclosure and the formation of a suction bubble through the opening), and
(ii) a collection system consisting of a tube joined to the top of the distal surface of the sealed enclosure, inside which a reservoir slides that is passed through by a perforation system oriented toward the mucosa, opposite the opening;
   wherein the walls of the sealed enclosure are hollow (to promote securing to the surface of the mucosa and to increase localized pressure for faster formation of the suction bubble and hence faster production of interstitial fluid while limiting the risk of overpressure of the mucosa (particularly the conjunctiva in the case of the eye) and optionally connected to a second suction system.

According to a particular embodiment of the device of the invention, the mucosa is the oral mucosa or the conjunctiva, preferably the conjunctiva.

According to a particular embodiment of the device of the present invention, the proximal surface of the enclosure is domed.

According to a particular embodiment of the device of the present invention, all or part of the proximal surface of the enclosure is covered with a flexible seal. Additionally, the part of the device in contact with the surface of the mucosa, e.g. the conjunctiva, can be covered with a membrane enabling epithelial (e.g. oral) or conjunctival impression collection.

According to a particular embodiment of the device of the present invention, the opening (or window) in the proximal surface of the enclosure has an area of between 3 and 350 mm², and is optionally adjustable.

According to a particular embodiment of the device of the present invention, the first suction system, and optionally the second suction system, creates a vacuum of less than about 300 mmHg.

According to a particular embodiment of the device of the present invention, the distal surface of the sealed enclosure is covered at least on its top with a material that can be pierced by the perforation system and closes over the latter when pierced to maintain the seal.

According to a particular embodiment of the device of the present invention, the sealed enclosure has transparent surfaces, and optionally a magnification means on the top of its distal surface.

According to a particular embodiment of the device of the present invention, the reservoir has a capacity ranging from 10 to 300 μl, preferably from 20 to 200 μl, more preferentially from 30 to 100 μl, most preferentially from 40 to 50 μl.

According to a particular embodiment of the device of the present invention, the length of the perforation system emerging from the reservoir is less than the maximum height of the sealed enclosure.

The present invention also relates to a method for collecting a sample of interstitial fluid from the mucosa of an animal for analysis, said method comprising:
placing a device according to the present invention on the surface of the mucosa at the collection site;
applying a vacuum to the walls and interior of the sealed enclosure so as to form a suction bubble at the opening of the proximal surface of the enclosure;
moving the reservoir of the collection system toward the mucosa until the perforation system passes through the distal surface of the sealed enclosure and the suction bubble;
collecting the interstitial fluid sample from the suction bubble in the reservoir.

According to a particular embodiment of the device of the invention, the mucosa is the oral mucosa or the conjunctiva, preferably the conjunctiva.

According to a particular embodiment of the method of the present invention, the vacuum applied in the walls and inside the sealed enclosure is less than 300 mmHg of mercury. According to a particular embodiment of the method of the present invention, the perforation system cannot descend beyond the proximal surface of the sealed enclosure.

### BRIEF DESCRIPTION OF THE FIGURES

FIG. 1 shows a cross sectional view of the enclosure, placed on an eye, and connected to a vacuum pump and pressure gauge. The window is an opening on the face of the enclosure (proximal surface) in contact with the surface of the eye (conjunctiva).

FIG. 2 shows a method of collecting interstitial fluid from the conjunctival surface of the eye passing through the window (bubble) via a device of the invention, at rest (FIG. 2A, off) and in action (FIG. 2B, on).

FIG. 3 shows the general principle of the enclosure of the device according to the invention.

FIG. 4 shows the operating sequence of a device according to the invention.

FIG. 5 shows a prototype ocular suction bell without the interstitial fluid collection device. (1) port to the suction system of the outer cylinder (3A) for holding the mucosal surface. (2) port to the suction system of the inner cylinder (3B) for collection of interstitial fluid or epithelial mucosal collection.

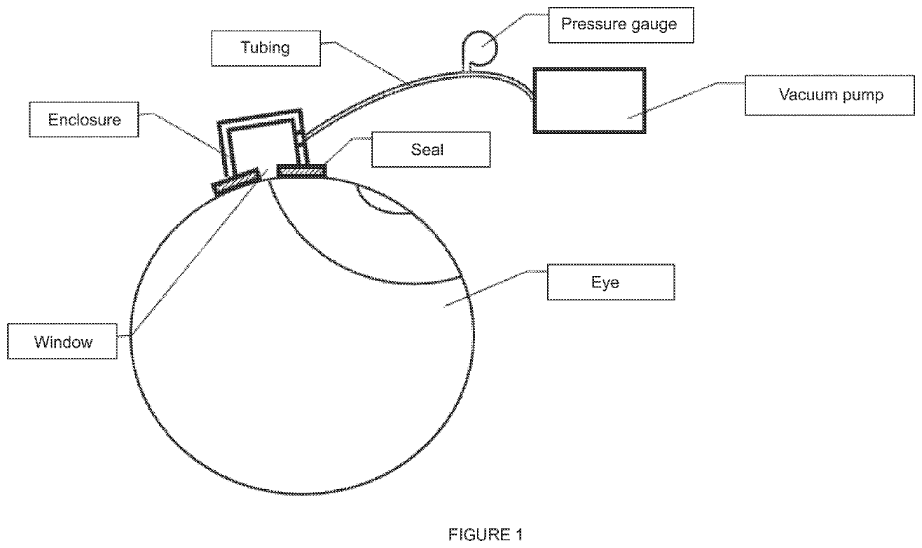

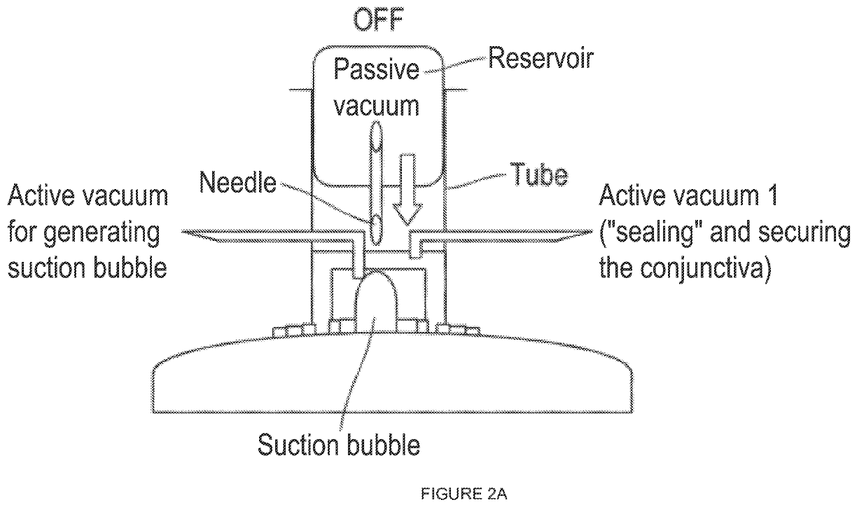

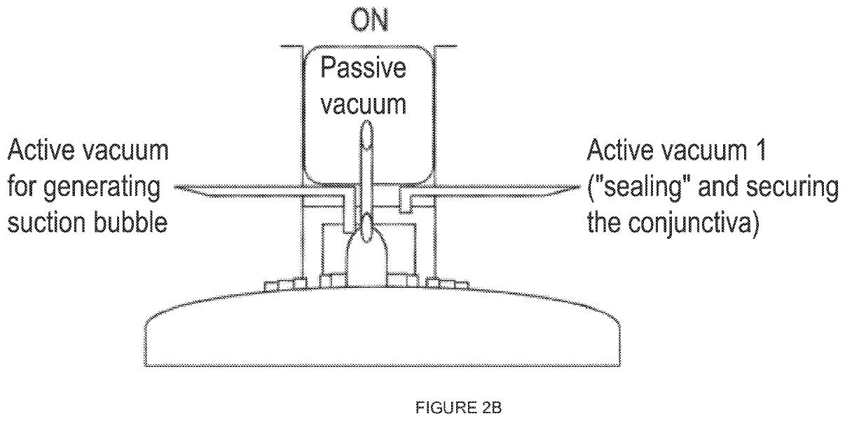

### EXAMPLES

### Example 1: Example Embodiments of a Device of the Invention and Implementation Thereof

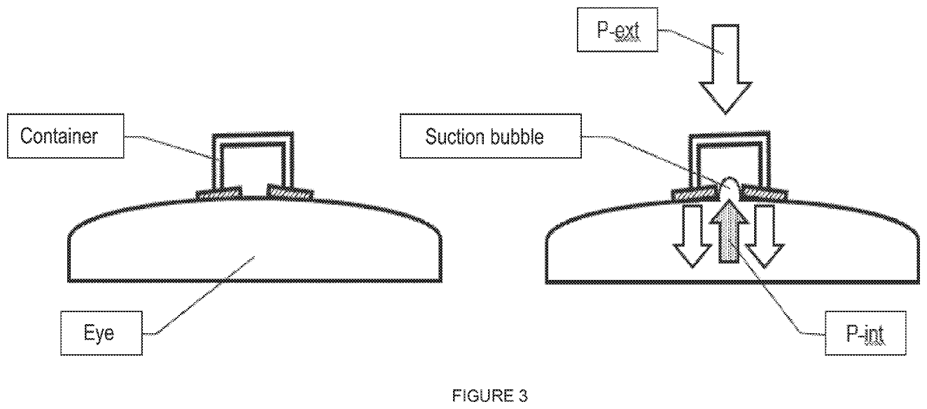

### General Principle of the Enclosure of the Device According to the Invention in Action on the Conjunctiva (FIG. 3)

On the left in FIG. 3, the enclosure of the device according to the invention is applied to the eye.

On the right in FIG. 3, the vacuum generated inside the enclosure has caused a suction “bubble”/outgrowth of the eye through the window, that is, a bubble whose surface is conjunctival tissue and whose interior is interstitial fluid.

Because of the pressure difference with the outside, the vacuum inside the enclosure will create a force (Pext) applied to the enclosure in the direction of the eye at the contact areas (seal) of the enclosure with the surface of the eye, and symmetrically, there will be an overpressure (Pint) from inside the eye at the window pushing toward the inside of the enclosure. The combination of these two opposing forces creates an outgrowth of the eye (that is, a bubble whose outer surface is conjunctival tissue and whose interior is interstitial fluid) through the window.

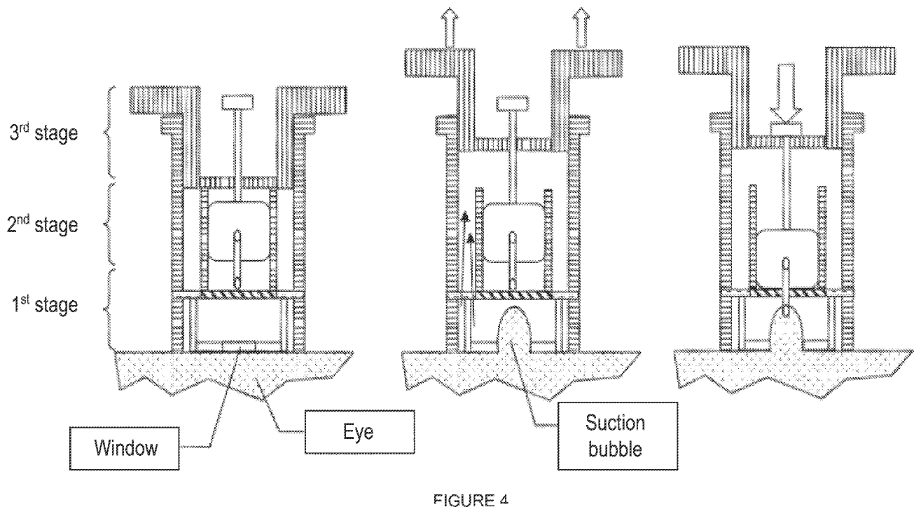

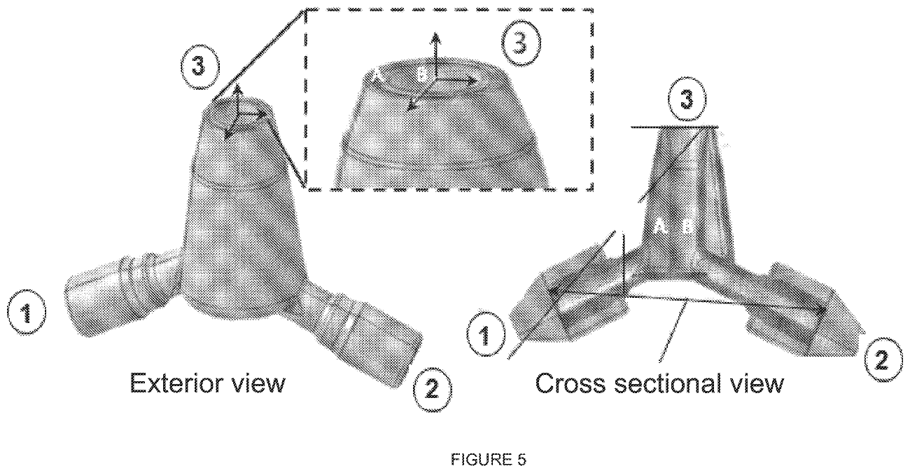

### Illustration of the Use of the Device According to the Invention on the Conjunctiva (FIG. 4)

The collection means, the vacuum means and the enclosure form part of the device according to the invention. The latter comprises a 3-stage tubular body with hollow walls. Its lower part, defining the 1^{st} stage, is applied to the conjunctiva, and has an upper face comprising a sealed central part and a peripheral part communicating with the walls of the 2^{nd} stage. This 2^{nd} stage comprises hollow walls of greater thickness than the walls of the 1^{st} stage, communicating with the walls and the interior of the 1^{st} stage. The center of this 2^{nd} stage features a tubular reservoir that is able to slide sealably along the walls, with a needle passing through its underside and an arm extending upwards. The pressure of the 2^{nd} stage is P2<atmospheric pressure. The 3^{rd} stage comprises ducts communicating with the walls of the 2^{nd} stage and, at its top, a sliding sealed slat, crossed at its center by the arm coming from the reservoir without air being able to pass through at this point.

The usage sequence for this device is as follows: after applying the bottom of the device (proximal surface) to the conjunctiva, the 3^{rd} stage sealing slat is slid so as to create a vacuum in the walls and inside the 1^{st} stage (via air communication between the walls of the 1^{st} and 2^{nd} stages and the ducts of the 3^{rd} stage). This vacuum, P1, creates a suction “bubble” on the surface of the eye, such that P2<P1. The arm connected to the reservoir is then pressed down, so that the needle extending from the reservoir passes through the upper surface (distal surface) of the 1^{st} stage and pierces the suction “bubble,” causing interstitial fluid to be collected. Once the collection has taken place, the device can be removed by lowering the 3^{rd} stage, which cancels out the vacuum.

On the left in FIG. 4, the device of the invention is affixed to the eye. The 1^{st} stage, used as an enclosure, has hollow walls and a window.

In the middle of FIG. 4, the raising of the 3^{rd} stage creates, via a network of hollow walls and ducts, a vacuum in the 1^{st} stage (interior and walls), which acts as an enclosure. This raising can be facilitated by handles that support the fingers of the operator, who holds the body of the device with the other hand. The suction bubble then forms.

On the right in FIG. 4, the pressure on the arm pushes the reservoir and needle through the suction bubble. Due to the pressure difference between the 1^{st} stage and the reservoir, the interstitial fluid contained in the suction bubble is drawn into the reservoir.

1. A device for collecting a sample of interstitial fluid from the mucosa of an animal for analysis, said device comprising:
(i) an airtight enclosure whose proximal surface in direct contact with the mucosa has an opening defining a collection area, and whose distal surface comprises at least one port connected to a first suction system, and
(ii) a collection system consisting of a tube joined to the top of the distal surface of the sealed enclosure, inside which a reservoir slides that is passed through by a perforation system oriented toward the mucosa, opposite the opening;
wherein the walls of the sealed enclosure are hollow, and optionally connected to a second suction system.

2. The device according to claim 1, where the mucosa is the conjunctiva.

3. The device according to claim 1, wherein the proximal surface of the enclosure is domed.

4. The device according to claim 1, wherein the proximal surface of the enclosure is covered by a flexible seal.

5. The device according to claim 1, wherein the opening in the proximal surface of the enclosure has an area of between 3 and 350 mm², and is optionally adjustable.

6. The device according to claim 1, wherein the suction system creates a vacuum of less than 300 mmHg.

7. The device according to claim 1, wherein the distal surface of the sealed enclosure is covered at least on its top with a material that can be pierced by the perforation system and closes over the latter when pierced to maintain the seal.

8. The device according to claim 1, wherein the sealed enclosure has transparent surfaces, and optionally a magnification means on the top of its distal surface.

9. The device according to claim 1, wherein the reservoir has a capacity ranging from 10 to 300 μl.

10. The device according to claim 1, wherein the length of the perforation system emerging from the reservoir is less than the maximum height of the sealed enclosure.
